(19) United States
(12) Patent Application Publication
(10) Pub. No.: US 2016/0143979 A1
(43) Pub. Date: May 26, 2016

(54) Long Pepper Extract an Effective Anticancer Treatment

(71) Applicants: Siyaram PANDEY, LaSalle (CA); Pamela Uzuazo OVADJE, Windsor (CA)

(72) Inventors: Siyaram PANDEY, LaSalle (CA); Pamela Uzuazo OVADJE, Windsor (CA)

(21) Appl. No.: 14/942,438

(22) Filed: Nov. 16, 2015

Related U.S. Application Data
(60) Provisional application No. 62/082,364, filed on Nov. 20, 2014.

(51) Int. Cl. A61K 36/67; A61K 45/06

(52) U.S. Cl. CPC A61K 36/67; A61K 45/06

(57) ABSTRACT

In a preferred embodiment, there is provided a method for preparing a medicament for the treatment or prevention of a cancer, the method comprising: grinding a Piper plant or a plant component thereof to obtain a ground plant mixture or powder; soaking the ground plant mixture or powder in a solvent to obtain a suspension having a liquid extract portion and a plant solid portion; and separating the liquid extract portion from the plant solid portion to provide a separated liquid extract for use in the medicament.

### RELATED APPLICATIONS

This application claims the benefit of 35 U.S.C. 119(e) to U.S. Provisional Patent Application Ser. No. 62/082,364 filed on 20 Nov. 2014.

### SCOPE OF THE INVENTION

The current invention relates to a method for preparing a medicament for the treatment or prevent of a cancer, and which broadly includes grinding a Piper plant or plant component to be extracted using a solvent, most preferably ethanol.

### BACKGROUND OF THE INVENTION

The continuing increase in the incidence of cancer signifies a need for further research into more effective and less toxic alternatives to current treatments. In Canada alone, it was estimated that 267,700 new cases of cancer will arise, with 76,020 deaths occurring in 2012 alone. The global statistics are even more dire, with 12.7 million cancer cases and 7.6 million cancer deaths arising in 2008. The hallmarks of cancer cells uncover the difficulty in targeting cancer cells selectively. Cancer cells are notorious for sustaining proliferative signaling, evading growth suppression, activating invasion and metastasis and resisting cell death among other characteristics. These characteristics pose various challenges in the development of successful anticancer therapies. The ability of cancer cells to evade cell death events has been the center of attention of much research, with focus centered on targeting the various vulnerable aspects of cancer cells to induce different forms of Programmed Cell Death (PCD) in cancer cells, with no associated toxicities to non-cancerous cells.

Apoptosis (PCD type I) has been studied for decades, the understanding of which will enhance the possible development of more effective cancer therapies. This is a form of cell death that is required for regular cell development and homeostasis, as well as a defense mechanism to get rid of damaged cells; cells undergoing apoptosis invest energy in their own demise so as not to become a nuisance. Cancer cells evade apoptosis in order to confer added growth advantage and sustenance, therefore current anticancer therapies endeavor to exploit the various vulnerabilities of cancer cells in order to trigger the activation of apoptosis through either the extrinsic or intrinsic pathways. The challenges facing some of the available cancer therapies are their abilities to induce apoptosis in cancer cells by inducing genomic DNA damage. Although this is initially effective, as they target rapidly dividing cells, they are usually accompanied by severe side effects caused by the non-selective targeting of normal non-cancerous cells, suggesting a need for other non-common targets for apoptosis induction without the associated toxicities.

Currently chemotherapy is limited mostly to genotoxic drugs that are associated with severe side effects due to non-selective targeting of normal tissue. Natural products play a significant role in the development of most chemotherapeutic agents, with 74.8% of all available chemotherapy being derived from natural products. Natural health products (NHPs) have shown great promise in the field of cancer research. The past 70 years have introduced various natural products as the source of many drugs in cancer therapy. Approximately 75% of the approved anticancer therapies have been derived from natural products, an expected statistic considering that more than 80% of the developing world's population is dependent on the natural products for therapy. Plant products especially contain many bioactive chemicals that are able to play specific roles in the treatment of various diseases. Considering the complex mixtures and pharmacological properties of many natural products, it becomes difficult to establish a specific target and mechanism of action of many NHPs. With NHPs gaining momentum, especially in the field of cancer research, there is a lot of new studies on the mechanistic efficacy and safety of NHPs as potential anticancer aunts. Long pepper, from the Piperaceae family, has been used for centuries for the treatment of various diseases. Several species of long pepper have been identified, including Piper Longum, Piper Belle, Piper Retrofactum, extracts of which have been used for years in the treatment of various diseases. A long list of uses and benefits are associated with extracts of different piper spp, with reports indicating their effectiveness as good digestive agents and pain and inflammatory suppressants. However, there is little to no scientific validation, only anecdotal evidence, for the benefits associated with the use of long pepper extracts. There are scientific studies have been carried out on several compounds present in extracts of long pepper, including piperines, which has been shown to inhibit many enzymatic drug bio-transforming reactions and plays specific roles in metabolic activation of carcinogens and mitochondrial energy production, and various piperidine alkaloids, with fungicidal activity.

### SUMMARY OF INVENTION

One possible non-limiting object of the present invention is to provide a method for preparing a medicament for the treatment or prevention of a cancer, and which includes as a main active ingredient substances derived from a readily available natural health product.

Another possible non-limiting object of the present invention is to provide a method for preparing a cancer medicament which does not strictly require inclusion of synthetic genotoxic drugs often associated with undesirable side effects and non-selective targeting of both cancerous and non-cancerous cells.

Another possible non-limiting object of the present invention is to provide a method for preparing a cancer medicament which may permit for more selective treatment of cancer cells, while reducing production costs.

In one aspect, the present invention provides a method for preparing a medicament for the treatment or prevention of a cancer, the method comprising: grinding a Piper plant or a plant component thereof to obtain a ground plant mixture or powder; soaking the ground plant mixture or powder in a solvent to obtain a suspension having a liquid extract portion and a plant solid portion; and separating the liquid extract portion from the plant solid portion to provide a separated liquid extract for use in the medicament.

In another aspect, the present invention provides a method for preparing a medicament comprising a Piper plant extract for treatment or prevention of a cancer, the method comprising the steps of: grinding a Piper plant seed to obtain a ground seed powder; steeping the ground seed powder in a solvent comprising ethanol to obtain a mixture having a liquid extract portion and a solid portion; separating the liquid extract portion from the solid portion, and removing the solvent from the liquid extract portion to obtain a solid or semi-solid extract; and optionally mixing the solid or semi-solid extract with a polar reconstitution solvent comprising dimethyl sulfoxide, water, alcohol or a mixture thereof, said alcohol preferably comprising one or more of n-butanol, isopropanol, n-propanol, ethanol and methanol.

In yet another aspect, the present invention provides a method of treating or preventing a cancer, the method comprising administering to a subject an effective amount of a medicament prepared by the method of the present invention.

To scientifically assess the anticancer potential of a preferred ethanolic extract of Long pepper (hereinafter also referred to as “PLX”), a plant of the Piperaceae family was selected with a view to assessing the efficacy of the anticancer mechanism of action of PLX against cancer cells. It has been recognized that the preferred ethanolic long pepper extract selectively induce caspase-independent apoptosis in cancer cells, without affecting non-cancerous cells, by targeting the mitochondria, leading to dissipation of the mitochondrial membrane potential and increase in reactive oxygen species or ROS production. Release of the AIF and endonuclease G from isolated mitochondria confirmed the mitochondria as a potential target of long pepper. The efficacy of PLX in in vivo studies indicates that oral administration may slow or even be able to halt the growth of colon cancer tumors in immunocompromised mice, with no associated toxicity. These results demonstrate the potentially safe and non-toxic alternative that is long pepper extract for cancer therapy.

It is to be appreciated that the Piper plant in its entirety or one or more plant components thereof may be utilized for preparing a medicament for treatment or prevention of a cancer. In one embodiment, the plant component comprises one or more of a seed, a leaf, a flower, a fruit, a root and a stem, or more preferably a seed.

In one embodiment, the solvent comprises one or more of water, pentane, cyclopentane, cyclohexane, benzene, toluene, 1,4-dioxane, chloroform, diethyl ether, dichloromethane, tetrahydrofuran, ethyl acetate, acetone, dimethylformamide, acetonitrile, dimethyl sulfoxide, propylene carbonate, formic acid, alcohol and acetic acid, wherein the alcohol preferably includes one or more of n-butanol, isopropanol, n-propanol, ethanol and methanol. In one embodiment, the solvent is ethanol.

In one embodiment, said soaking or steeping step comprises soaking or steeping the ground plant mixture or powder with or without shaking in the solvent for between about 5 minutes and about 72 hours, preferably between about 4 hours and about 48 hours, more preferably between about 12 hours and about 36 hours, or most preferably for about 24 hours, and at a temperature between about 0° C. and about 100° C., preferably between about 10° C. and about 75° C., more preferably between about 15° C. and about 50° C., or most preferably about 25° C.

It is contemplated further process improvements in the extraction method may be implemented. These may, for example, include: cryogenic grinding of the plant material to for example micronized or nano scale particles; harvesting the long pepper fruit when the active compounds are at their highest concentrations; and better storage of long pepper between harvest and process to preserve the active compounds at their optimum levels.

In one embodiment, the method further comprises freezing the Piper plant or the plant component to obtain a frozen plant stock, said freezing step being selected to effect at least partial disruption of one or more plant cells, and wherein said grinding step comprises dry grinding the frozen plant stock to obtain the ground plant powder, wherein during said dry grinding step, the frozen plant stock is maintained at a grinding temperature below about 40° C., preferably below about 0° C., more preferably below about −25° C. or most preferably below about −40° C. Preferably, said freezing step comprises contacting or submerging the Piper plant or the plant component in liquid nitrogen, or freezing the Piper plant or the plant component to an average freezing temperature between about −210° C. and about −30° C. In one embodiment, said dry grinding step comprises dry grinding the frozen plant stock to an average particle size of less than about 100 μm, preferably less than about 70 μm or more preferably less than about 45 μm.

In one embodiment, prior to said freezing step, the method further comprises drying said Piper plant or the plant component to a relative humidity between about 5% and about 10%.

In one embodiment, said dry grinding step comprises dry grinding the frozen plant stock with a grinder selected from the group consisting of a pulverizer, an impingement grinder and a micronized milling machine, and wherein the grinder or a component thereof is cooled below about −25° C. or preferably below about −50° C., to prevent heating on contact with the frozen plant stock or the ground plant powder. In one embodiment, the grinder or a component thereof is cooled by directly or indirectly contacting with liquid nitrogen. In one embodiment, the grinder defines a grinding chamber sized for receiving the frozen plant stock, said method further comprising flowing or adding liquid nitrogen to the grinding chamber during said dry grinding the frozen plant stock in the grinding chamber. In one embodiment, said dry grinding step further comprises straining the ground plant powder from the grinder through a sieve sized to obtain a sieved ground plant powder having an average particle size of less than about 100 μm, preferably less than about 70 μm or more preferably less than about 45 μm. The applicant has appreciated that such dry grinding to obtain the sieved ground plant powder may permit for improved extraction of active ingredients from the Piper plant, while reducing loss of bioactivity.

In one embodiment, said separation step comprises of filtration, wherein said filtration is performed once or more than once using a plurality of filters of same or different pore sizes. Preferably, said separation step comprises filtering the suspension at least twice with a paper filter having a particle retention greater than about 20 μm.

It is to be appreciated that the liquid extract portion may be subject to further processing. In one embodiment, said method further comprises removing the solvent from the separated liquid extract to obtain a solid or semi-solid extract, and optionally mixing the extract with one or more of a pharmaceutically acceptable carrier, a diluent, a binding agent, an adjuvant and an anticancer aunt. The solid or semi-solid extract may be administered to a subject. In one embodiment, the carrier comprises one or more of a polar reconstitution solvent and a buffer solution, the reconstitution solvent preferably comprising dimethyl sulfoxide, alcohol or a mixture thereof, the alcohol preferably comprising one or more of n-butanol, isopropanol, n-propanol, ethanol and methanol, and the buffer solution preferably comprising a phosphate buffered saline solution or a sodium bicarbonate buffered saline solution. In one embodiment, the anticancer agent comprises metformin, hydroxyurea, cyclophosphamide, etoposide or another anticancer natural extract.

It is to be appreciated that the Piper plant species for use with the current invention is not intended to be specifically limited to Piper longum, and may alternatively include among others Piper belle, Piper retrofactum and Piper nigrum. Indeed, the applicant has appreciated that the method may be practiced with a Piper plant other than Piper longum, while retaining anticancer activity of the medicament. Preferably, the Piper plant is selected to provide in the liquid extract portion or the medicament two or more of dihydropiperlongumine, piperlongumine, dihydropiperlonguminine, piperlonguminine, piperinine, piperazine, piperidine and piperine, or preferably two or more of dihydropiperlongumine, piperlongumine, dihydropiperlonguminine, piperlonguminine, piperinine and piperine. The applicant has appreciated that the aforementioned compounds may provide for anticancer activities.

The medicament of the current invention may permit for treatment or prevention of a cancer, including but not limited to colorectal cancer, ovarian cancer, pancreatic cancer, melanoma, breast cancer, osteosarcoma, lung cancer, prostate cancer, glioblastoma, lymphoma or leukemia, or preferably colorectal cancer, ovarian cancer, pancreatic cancer, melanoma, glioblastoma or leukemia, wherein the leukemia, includes T cell leukemia, acute myeloid leukemia, chronic myeloid leukemia, chronic myelomonocytic leukaemia, chronic lymphocytic leukemia or acute lymphoblastic leukemia. Some clinical studies have been conducted to evaluate anticancer potential or activities of preferred medicaments of the present invention, including those for glioblastoma.

In one embodiment, the method further comprises freezing the Piper plant seed to obtain a frozen seed stock, and wherein said grinding step comprises dry grinding the frozen seed stock to obtain the ground seed powder, wherein during said dry grinding step, the frozen seed stock is maintained at a grinding temperature below about 0° C., preferably below about −25° C. or more preferably below about −40° C. Preferably, said freezing step comprises contacting or submerging the Piper plant seed in liquid nitrogen, or freezing the Piper plant seed to an average freezing temperature between about −210° C. and about −30° C. Preferably, said dry grinding step comprises dry grinding the frozen seed stock to an average particle size of less than about 100 μm. preferably less than about 70 μm or more preferably less than about 45 μm with a grinder selected from the group consisting of a pulverizer, an impingement grinder and a micronized milling machine, wherein the grinder or a component thereof is cooled below about −25° C. or preferably below about −50° C., to prevent heating on contact with the frozen seed stock or the ground seed powder.

In one embodiment, the grinder or a component thereof is cooled by directly or indirectly contacting with liquid nitrogen. In one embodiment, the grinder defines a grinding chamber sized for receiving the frozen plant stock, said method further comprising flowing or adding liquid nitrogen to the grinding chamber during said dry grinding the frozen plant stock in the grinding chamber. In one embodiment, said dry grinding step further comprises straining the ground plant powder from the grinder through a sieve sized to obtain a sieved ground plant powder having an average particle size of less than about 100 μm, preferably less than about 70 μm or more preferably less than about 45 μm. The applicant has appreciated that such dry grinding to obtain the sieved ground plant powder may permit for improved extraction of active ingredients from the Piper plant, while reducing loss of bioactivity.

In one embodiment, said mixing step comprises mixing the solid or semi-solid extract with the polar reconstitution solvent to obtain a reconstituted extract, and mixing the reconstituted extract with a buffer solution and optionally an anticancer agent, the buffer solution preferably comprising a phosphate buffered saline solution or a sodium bicarbonate buffered saline solution, and the anticancer agent preferably comprising metformin, hydroxyurea, cyclophosphamide or etoposide.

### BRIEF DESCRIPTION OF THE DRAWINGS

Reference may now be had to the following detailed description taken together with the accompanying drawings which:

FIG. 1 shows four (4) bar graphs illustrating absorbance values at 450 nm expressed as a percent of the control for colon (HCT116), ovarian (OVCAR-3), pancreatic (BxPC-3) cancer and Melanoma (G-361) cells treated with a crude ethanolic extract of long pepper at indicated concentrations, and then incubated with WST-1 cell viability dye for 4 hours. The values are expressed as mean±SD from quadruplicates of 3 independent experiments, with **P<0.0001;

FIG. 2 shows ten (15) bar graphs illustrating the results of image-based cytometry for induction of cell death in respect of human pancreatic (BxPc-3) cancer and T cell leukemia cells treated with PLX at indicated concentrations, and which were subsequently incubated with propidium iodide at indicated time points;

FIG. 3 shows six (6) images of human colon cancer cells (HT-29) treated with PLX at indicated concentrations, and subsequently incubated with propidium iodide, and which are obtained from fluorescence microscopy (at 400× magnification on a fluorescent microscope, scale bar=15 μm) for assessing induction of cell death as characterized by presence of propidium iodide positive cells;

FIG. 4 shows six (6) images of normal colon epithelial cells (NCM460) treated with PLX at indicated concentrations, and subsequently incubated with propidium iodide, and which are obtained from fluorescence microscopy (at 400× magnification on a fluorescent microscope, scale bar=15 μm) for assessing induction of cell death as characterized by presence of propidium iodide positive cells;

FIG. 5 shows nineteen (19) bar graphs illustrating the results of image-based cytometry for apoptotic induction (% annexin V positive) and necrosis (% PI positive) in respect of E6-1 cells following PLX treatment at indicated concentrations, with the lack of annexin V or PI staining indicating live cells following the treatment (% annexin V/PI negative cells, *P<0.05, **P<0.003, ***P<0.0001);

FIG. 6 shows nineteen (19) bar graphs illustrating the results of image-based cytometry for apoptotic induction (% annexin V positive) and necrosis (% PI positive) in respect of HT-29 cells following PLX treatment at indicated concentrations, with the lack of annexin V or PI staining indicating live cells following the treatment (% annexin V/PI negative cells, *P<0.05, **P<0.003, ***P<0.0001);

FIG. 7 shows eight (8) bar graphs illustrating the results of image-based cytometry for detection of DNA fragmentation with TUNEL labeling in respect of cancer cells treated with PLX and VP16 (as a positive control for DNA damage), and which were subsequently labelled with DNA staining solution and quantified by image-based cytometry (treated cells were compared to the control untreated cell sample, ***P<0.0001);

FIG. 8 shows eighteen (18) images of OVCAR-3, G-361 and NCM460 cells obtained with a fluorescent microscope at 400× magnification (scale bar=15 μm), and which were treated with PLX at indicated concentrations and stained with Hoechst to characterize nuclear morphology and Annexin-V to detect apoptotic cells;

FIG. 9 shows eight (8) images of HT-29 and NCM460 cells obtained with a fluorescent microscope at 400× magnification (scale bar=15 μm), and which were treated with PLX at indicated concentrations and stained with Hoechst to characterize nuclear morphology and subject to phase contrast microscopy for cellular morphology;

FIG. 10 shows two (2) bar graphs illustrating percent viability values for HT-29 colorectal cancer cells and non-cancerous NCM460 cells treated with PLX and subsequently incubated with WST-1 cell viability dye for 4 hours, and which were obtained by measuring absorbance values at 450 nm and expressed as a percent of the control (values are expressed as mean±SD from quadruplicates of 3 independent experiments. **P<0.0001);

FIG. 11 shows a bar graph illustrating fluorescent readings (an average of 6 readings per well and a minimum of three wells per experiment, and the average of three independent experiments shown) expressed as activity per μg of protein (in fold), and which are obtained with a spectrofluorometer in respect of cell lysate of BxPc-3 cells incubated with caspase substrates specific to each caspase 3, 8 or 9 for an hour, where the BxPc-3 cells were treated with 0.10 mg/ml PLX at indicated time points, collected, washed and incubated with lysis buffer;

FIG. 12 shows two (2) bar graphs illustrating absorbance values at 450 nm expressed as a percent of the control in respect of HCT 116 and BxPC-3 cancer cells pretreated or not pretreated with Z-VAD-fink for an hour before PLX treatment at indicated concentrations, and which are representative of a WST-1 cell viability assay of the cancer cells (the values are expressed as mean±SD from quadruplicates of 3 independent experiments. **P<0.0001);

FIG. 13 shows nine (9) bar graphs illustrating fluorescence results obtained with an image based cytometer in respect of colon cancer (HT-29), normal colon epithelial (NCM460) and normal human fibroblast (NHF) cells treated with PLX at indicated concentrations for 48 hours, and subsequently treated with H₂DCFDA;

FIG. 14 shows a bar graph illustrating quantified results of the fluorescence results shown in FIG. 13 using Graphpad prism 6.0;

FIG. 15 shows a bar graph illustrating absorbance values at 450 nm expressed as a percent of the control in respect of HCT 116 colon cancer cells treated with 3 mM N-acetylcysteine for an hour, then with PLX at indicated concentrations for 72 hours and being subject a WST-1 assay (the values are expressed as mean±SD from quadruplicates of 3 independent experiments. **P<0.05);

FIG. 16 shows eight (8) bar graphs illustrating fluorescence values obtained using image based cytometry in respect of colon cancer (HT-29), ovarian cancer (OVCAR-3) and normal colon epithelial (NCM460) cells treated with PLX at indicated concentrations for 48 hours, and subsequently incubated with JC-1;

FIG. 17 shows a bar graph illustrating quantified results of the fluorescence results shown in FIG. 16;

FIG. 18 shows eight (8) images of OVCAR-3 and NCM460 cells taken at 400× magnification using a fluorescent microscope (scale bar=15 μm), and which were treated with PLX, and subsequently incubated with TMRM cationic mitochondrial membrane permeable dye (corresponding Hoechst dye images are also shown);

FIG. 19 shows on the left hand portion a series of images obtained from western blot analyses for pro-apoptotic factors AIF and EndoG in mitochondrial supernatants obtained by centrifuging samples of isolated mitochondria of OVCAR-3 cells treated directly with PLX or solvent control (ethanol) for 2 hours (the mitochondrial pellets were probed for SDHA to serve as loading controls, and the images are representative of 3 independent experiments demonstrating similar trends), and on the left hand portion two (2) bar graphs showing ratios of AIF or EndoG to SDHA (the values are expressed as mean±SD of quadruplicates of 1 independent experiment; *p<0.01 versus solvent control (ethanol));

FIG. 20 shows a bar graph (left) and a table (right) illustrating results from a protein urinalysis by Bradford assay and dipstick analysis conducted in respect of BALB/C mice divided into three groups, or namely a control group (3 animals, untreated and given plain filtered water), a gavage control group (3 animals, given 50 mg/kg/day vehicle (DMSO) and a treatment group (4 animals, given 50 mg/kg/day PLX);

FIG. 21 shows a line graph illustrating weight changes of the BALB/c mice detailed above in respect of FIG. 20;

FIG. 22 shows six images of hematoxylin and eosin stained tissue sections of the liver, heart and kidney of the BALB/c mice detailed above in respect of FIG. 20, and which were obtained on a bright field microscope at 63× objective;

FIG. 23 shows nine (9) photographs illustrating representative tumor sizes on CD-1 nu/nu mice subcutaneously injected with colon cancer cells (HT-29 (p53^{-/-}) on the left flank and HCT116 (p53^{+/+}) on the right flank), and which were divided into three groups, or namely a control group, a gavage control group and a treatment group;

FIG. 24 shows two (2) line graphs illustrating average body weights and tumor volumes of the CD-1 nu/nu mice detailed above in respect of FIG. 23 over time;

FIG. 25 shows twenty (20) images illustrating histopathological analysis of tissue samples obtained from the CD-1 nu/nu mice detailed above in respect of FIG. 23, and which represents hematoxylin and eosin stained tissue sections of the livers, hearts, kidneys and tumors (the images were obtained on a bright field microscope at 10× and 63× objective); and

FIG. 26 shows two (2) chromatograms of a piperamides standard mix (1 mg/mL at 1 μL/standard) and a PLX (10 mg/mL at 2 μL/sample).

### DETAILED DESCRIPTION OF THE PREFERRED EMBODIMENTS

Indian long pepper seeds were obtained from Quality Natural Foods limited, Toronto Ontario. The plant material was ground up and extracted in anhydrous ethanol (100%) in a ratio of about 1:10. The extraction was carried out overnight on a shaker at room temperature. The extract was passed through a P8 coarse filter, followed by a 0.45 μn1 filter. The solvent was evaporated using a rotary evaporator at 40° C. resulting in a residual powdered concentrate. This powder concentrate was then reconstituted in dimethylsulfoxide (Me₂SO) at a stock concentration of approximately 450 mg/ml. When administered the reconstituted solution is mixed with phosphate buffered saline solution and given orally. The applicant has recognized that Long pepper extracts may represent a new NHP, with better selective efficacy against cancer cells.

Alternatively, a preferred long pepper extract may be prepared with whole Piper longum or seeds thereof purchased from Premier Herbal Inc. of Toronto, Ontario, and which originate from India.

The applicant has examined the efficacy of an ethano lie extract of Long Pepper against various cancer cells, and has attempted to elucidate the mechanism of action, following treatment. Results from this preliminary studies suggest that PLX may reduce the viability of various cancer cell types in a dose and time dependent manner, where apoptosis induction was observed, following mitochondrial targeting. Due to the low doses of PLX required to induce apoptosis in cancer cell a therapeutic window of this extract is furthermore suggested. Preliminary studies suggest the induction of apoptosis may be caspase-independent, although there was activation of both the extrinsic and intrinsic pathways and the production of ROS was not essential to the mechanism of cell death induction by PLX. The ability of PLX to target multiple vulnerabilities of cancer cells and still act to induce apoptosis in the presence of different types of inhibitors suggests the potential application of PLX in safe and efficacious cancer therapy.

In one experiment, following treatment with ethanolic long pepper extract, cell viability was assessed using a water-soluble tetrazolium salt; apoptosis induction was observed following nuclear staining by Hoechst, binding of annexin V to the externalized phosphatidyl serine and phase contrast microscopy. Image-based cytometry was used to detect the effect of long pepper extract on the production of reactive oxygen species and the dissipation of the mitochondrial membrane potential following Tetramethylrhodamine or 5,5,6,6′-tetrachloro-1,1′,3,3′-tetraethylbenzimidazolylcarbocyanine chloride staining (JC-1). Assessment of PLX in vivo was carried out using Balb/C mice (toxicity) and CD-1 nu/nu immunocompromised mice (efficacy). HPLC analysis enabled detection of some primary compounds present within our long pepper extract. Without being bound by a particular theory, preliminary testing results suggest that an ethanolic long pepper extract may selectively induce caspase-independent apoptosis in cancer cells, without affecting non-cancerous cells, by targeting the mitochondria, leading to dissipation of the mitochondrial membrane potential and increase in ROS production. Release of the AIF and endonuclease G from isolated mitochondria confirms the mitochondria as a potential target of long pepper. The efficacy of PLX in in vivo studies indicates that oral administration may be able to retard or halt the growth of colon cancer tumors in immunocompromised mice, with no associated toxicity. These results suggest the potentially safe and non-toxic alternative that is long pepper extract for cancer therapy.

To further explicitly illustrate the effectiveness of the medicament of the present invention, detailed descriptions of exemplary experiments are provided below:

### i) Ethanolic Extract of Long Pepper Effectively and Selectively Reduces the Viability of & Induces Apoptosis in Cancer Cells in a Dose & Time Dependent Manner

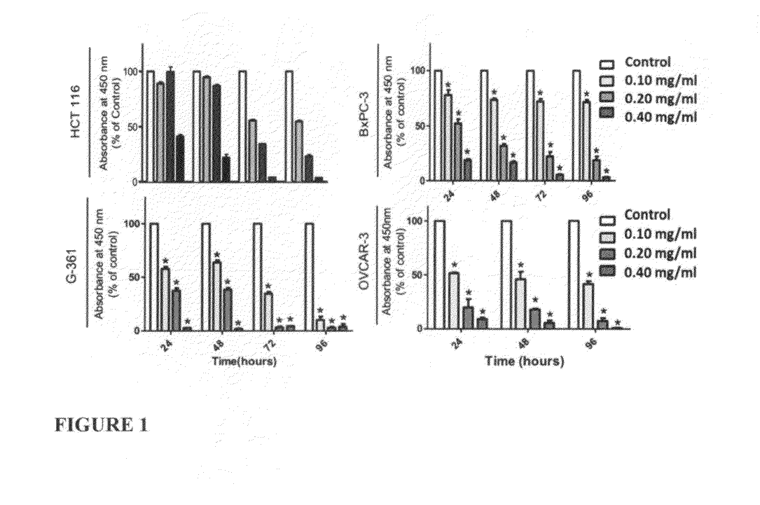

In one experiment, as a first step in assessing the effect of long pepper extract the effect of PLX on the viability of cancer cells was assessed. In particular, following treatment with increasing concentration of PLX at increasing time points, cells were incubated with a water soluble tetrazolium salt, which gets metabolized to a red formazan product by viable cells with active metabolism. This product can then be quantified by absorbance spectrometry. The efficacy of crude PLX in reducing the viability of cancer cells was observed, including colon (HCT116), pancreatic (BxPC-3), ovarian cancer (OVCAR-3) and melanoma cells. This effect was dose and time dependent (see FIG. 1).

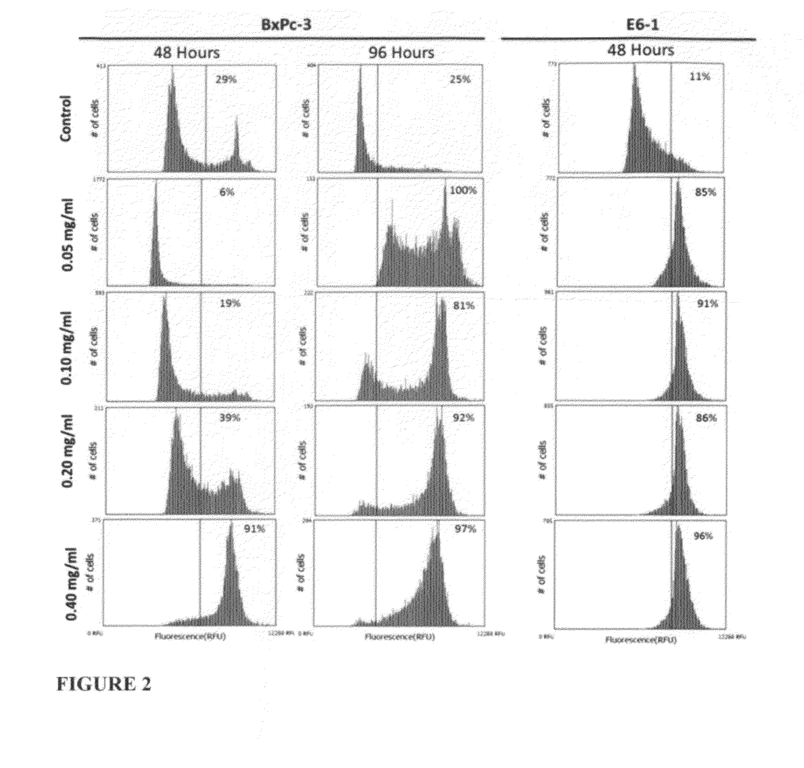

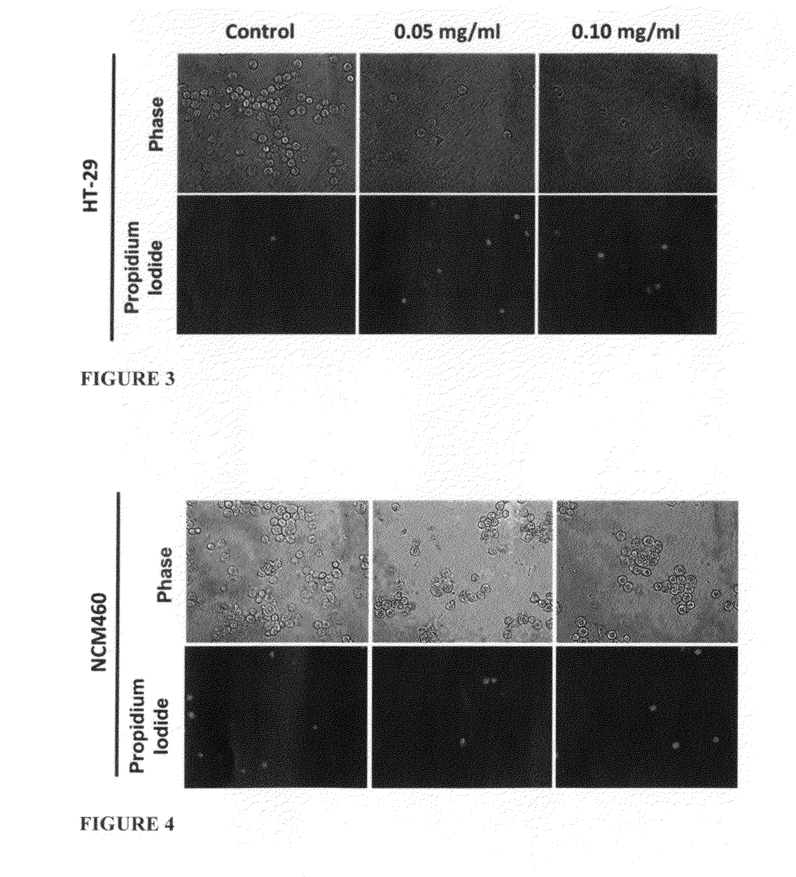

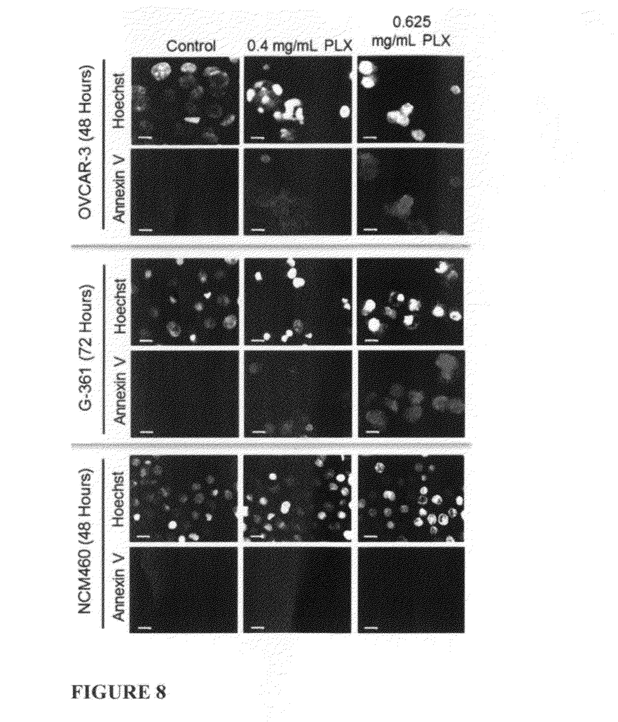

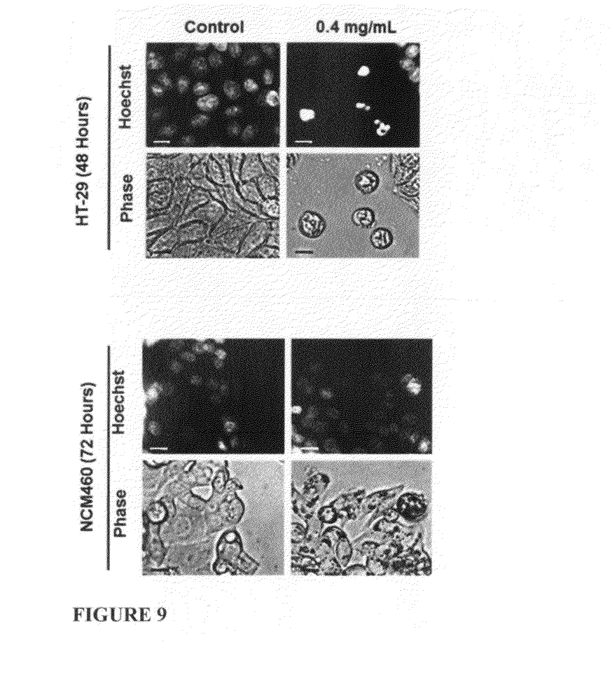

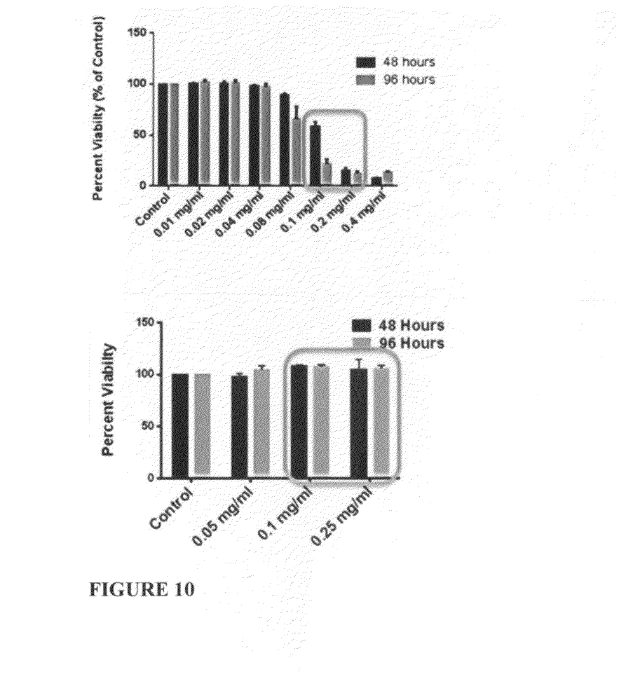

To further evaluate the anticancer activity of PLX, its role in cell death and its selectivity to cancer cells was assessed. The preliminary results demonstrate that PLX may be able to selectively induce cell death in cancer cells (colon, pancreatic and leukemia) in a dose and time dependent manner, as characterized by the increase in propidium iodide positive cells in cancer cells treated with PLX (see FIGS. 2 and 3). Furthermore, this effect was selective, as normal colon epithelial cells remained substantially unaffected by this treatment, at the same concentrations and time-points (see FIGS. 3 and 4). Additionally, apoptosis induction in various cancer cells, melanoma (G-361), ovarian and colon cancer (HT-29) cells, was confirmed by Annexin-V binding assay. This induction of apoptosis was confirmed to be selective to cancer cells, as normal colon cells (NCM460) remained unaffected by PLX treatment. This was indicated by nuclear condensation, cell morphology and externalization of phosphatidyl serine to the outer leaflet of the cell membrane, as indicated by Hoechst staining, phase contrast images and binding of annexin V dye respectively (see FIGS. 8 to 10). The selectivity of PLX to cancer cells was further confirmed by the WST-1 cell viability assay that showed that PLX was highly effective at such low doses, a therapeutic window was easily observed (see FIG. 10). Treatment of HT-29 with 0.20 mg/ml effectively reduced the viability by approximately 90%, while NCM460 cells remained at 100% viability at the same dose. This indicates that PLX can be more effective at very low doses, further reducing the chances of toxicity associated with treatment.

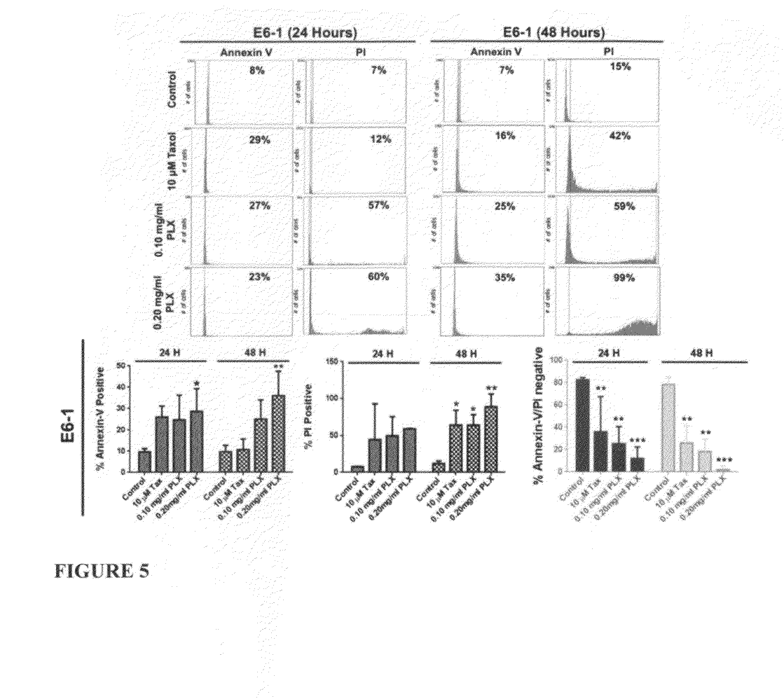

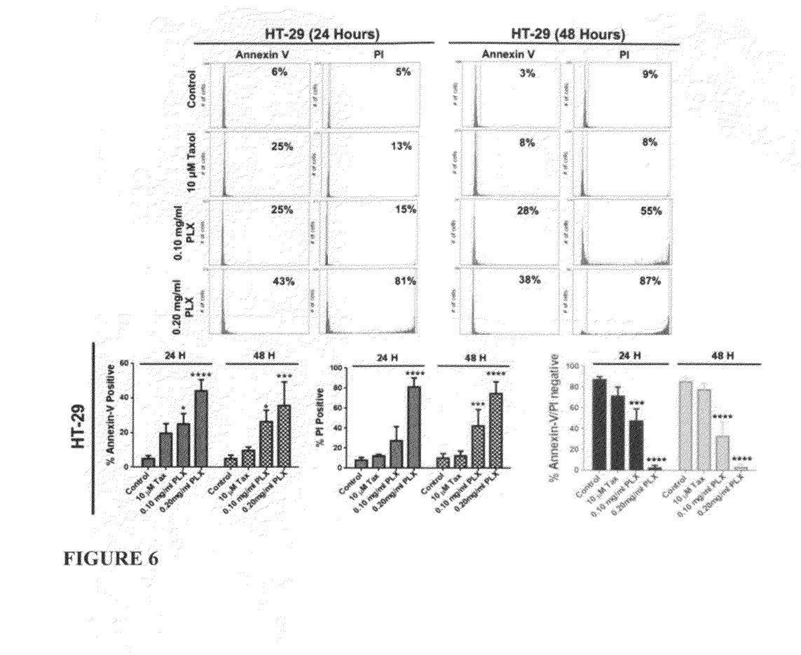

To confirm the induction of apoptosis, the binding of Annexin V to externalized phosphatidylserine on the outer cellular surface, was assessed. Following treatment with PLX, cells (FIG. 5—E6-1 and FIG. 6—HT-29 cells) were washed twice in phosphate buffer saline (PBS). Subsequently, cells were resuspended and incubated in Annexin V binding buffer (10 mM HEPES, 10 mM NaOH, 140 mM NaCl, 1 mM CaCl2, pH 7.6) with Annexin V Alexa Fluor-488 (1:50) (Invitrogen, Canada, Cat No. A13201) for 15 minutes. In the final 10 minutes of incubation, 1 mg/ml propidium iodide was added to the microcentrifuge tube and incubated for the final 10 minutes in the dark. Image based cytometry was used to quantify the percentage of programmed cell death (annexin V positive cells) and necrotic cell death (propidium iodide positive cells) occurring after treatment (see FIGS. 5 and 6).

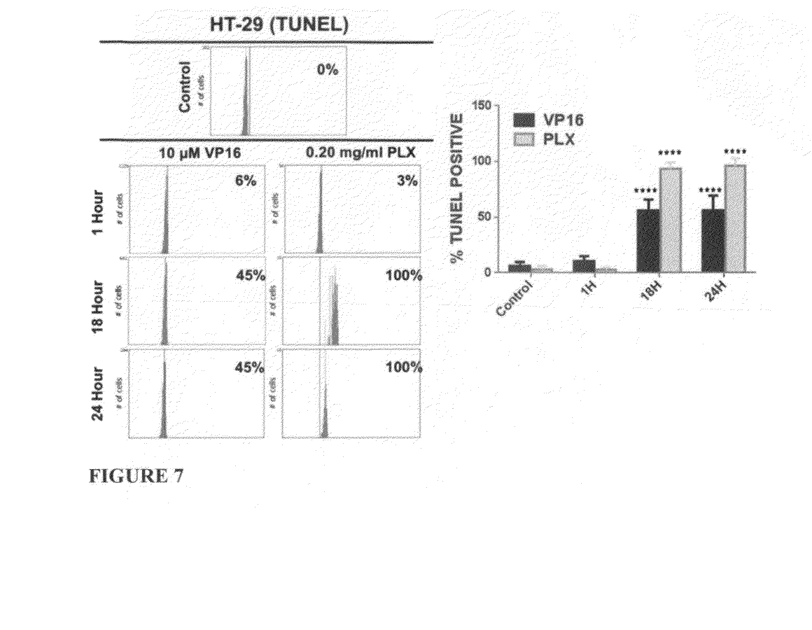

Following PLX treatment, HT-29 cells were labeled with the Terminal deoxynucleotidyl transferase dUTP nick end labeling (TUNEL) assay. The assay was performed according to the manufacturer's protocol (Molecular Probes, Eugene, Oreg.), in order to detect DNA damage Cells were treated with PLX or VP-16 (as a positive control) at indicated concentrations and time points and analyzed for the fragmentation of DNA. Following treatment, cells were fixed by suspending them in 70% (v/v) ethanol and stored at −20° C. overnight. The sample was then incubated with a DNA labeling solution (10 μL reaction buffer, 0.75 μL TdT enzyme, 8 μL BrdUTP, 31.25 μL of dH₂O) for 1 hour at 25° C. Each sample was exposed to an antibody solution (5 μL Alexa Fluor 488 labeled anti-BrdU antibody and 95 μL rinse solution). The cells were incubated with the antibody solution for 20 minutes and TUNEL positive cells were quantified by image-based cytometry (see FIG. 7).

### ii) PLX Induces Caspase-Independent Apoptosis in Human Cancer Cells

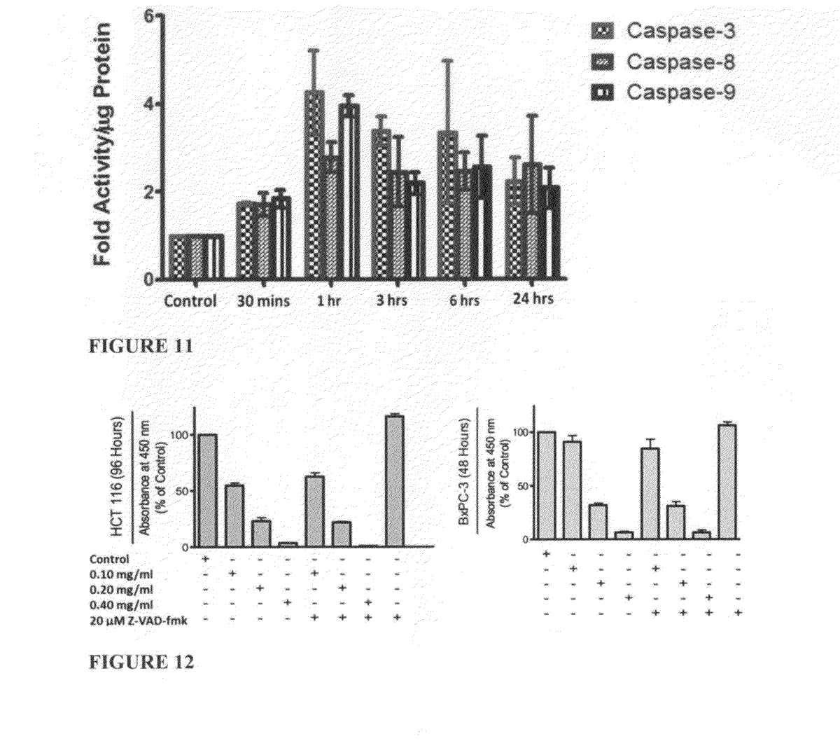

It has been recognized that caspases are cysteine aspartic proteases that play a predominant role as death proteases. Their roles in various cell death processes remains controversial, as their activation or inhibition could be essential to the progression of inhibition of cell death pathways. In another experiment, to assess the role of caspases, following treatment with 0.10 mg/ml PLX, at indicated time points, BxPc-3 cells were collected, washed and incubated with lysis buffer to obtain cell lysate. The cell lysate was incubated with caspase substrates, specific to each caspase (3, 8 and 9) and incubated for an hour. Fluorescence readings were obtained using a spectrofluorometer. Results indicate that PLX is able to activate both pathways (extrinsic and intrinsic apoptosis) in a time dependent manner. This was observed as rapid activation of caspases-3, 8 and 9 were observed as early as an hour, following treatment (see FIG. 11). To determine the importance of these activated caspases to the apoptosis-inducing effect of PLX, colon (HCT116) and pancreatic (BxPc-3) cancer cells were pre-treated with a pan-caspase inhibitor, Z-VAD-fink (20 μM), for an hour before treatment with PLX. Following treatments, the VST-1 cell viability assay was used to assess for viability and efficacy of PLX. Our results indicate that the inhibition of caspases may not prevent the reduction of viability (see FIG. 12), suggesting that the effect of PLX in cancer cells may be caspase independent.

iii) Long Pepper Extract Induces Oxidative Stress and Targets the Mitochondria of Cancer Cells

Generation of oxidative stress has been well established as a major player in the induction of several cell death processes, especially apoptosis. In another experiment, the applicant has also examined the role of oxidative stress in PLX induced apoptosis. Following treatment with PLX for 48 hours, cells were incubated with 2′,7′-Dichlorofluorescin diacetate H₂DCFDA for 45 minutes. The resulting green fluorescence histograms were obtained using a TALI image-based cytometer.

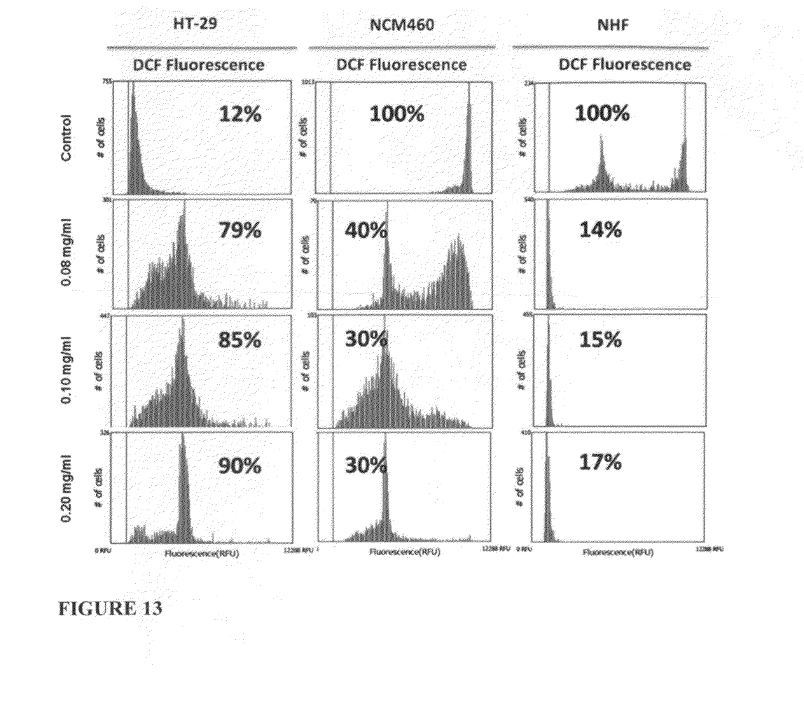

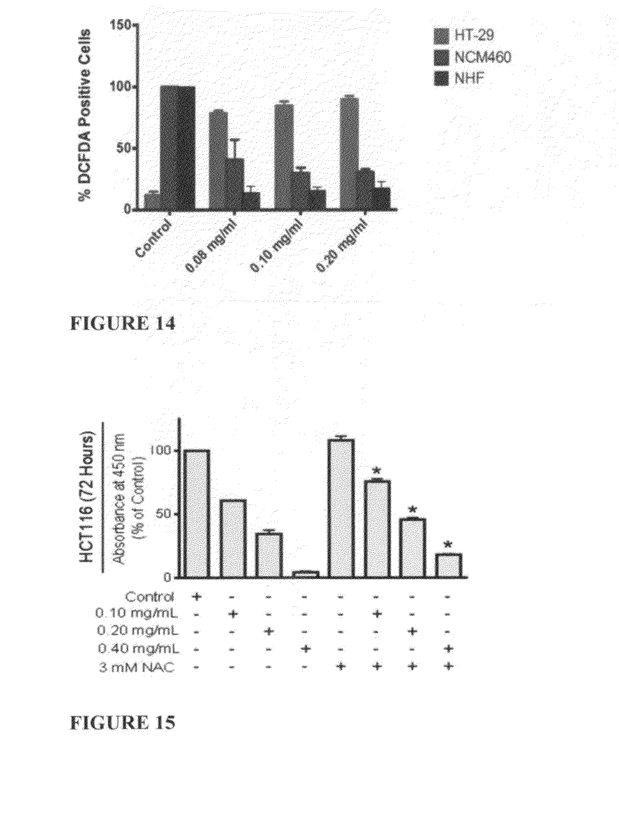

From the results, it was observed that PLX induced extensive generation of whole cell reactive oxygen species (ROS) in HT-29 colon cancer cells, while acting to suppress any ROS present in the non-cancerous cell lines, NCM460 and normal human fibroblasts (NHF) (see FIGS. 13 and 14). This appears to confirm the results of selectivity and indicates that PLX might act as a pro-oxidant in cancer cells in order to induce apoptosis.

To determine if this oxidative stress was essential to PLX activity, HCT116 colon cancer cells were pre-treated with N-acetyl-L-cysteine (NAC), a well-established anti-oxidant, used extensively in vitro studies, before treatment with PLX. Subsequent to PLX treatment, cells were analyzed for effect of PLX on viability, using the WST-1 viability assay. The results suggest that although PLX may act to induce oxidative stress to cause apoptosis, this oxidative stress is not essential to its activity. Both the cells treated with PLX alone and NAC followed by PLX showed a reduction in their viability (see FIG. 15).

The mitochondria have also been shown to play a major role in the progression and execution of apoptosis. The permeabilization of the mitochondrial membrane usually leads to the release of pro-apoptotic factors, including cytochrome c, apoptosis inducing factor (AIF) and endonuclease G (EndoG). These factors cause a caspase-independent pathway for apoptosis to pass through and could bypass the antioxidant effects of NAC observed in FIG. 15.

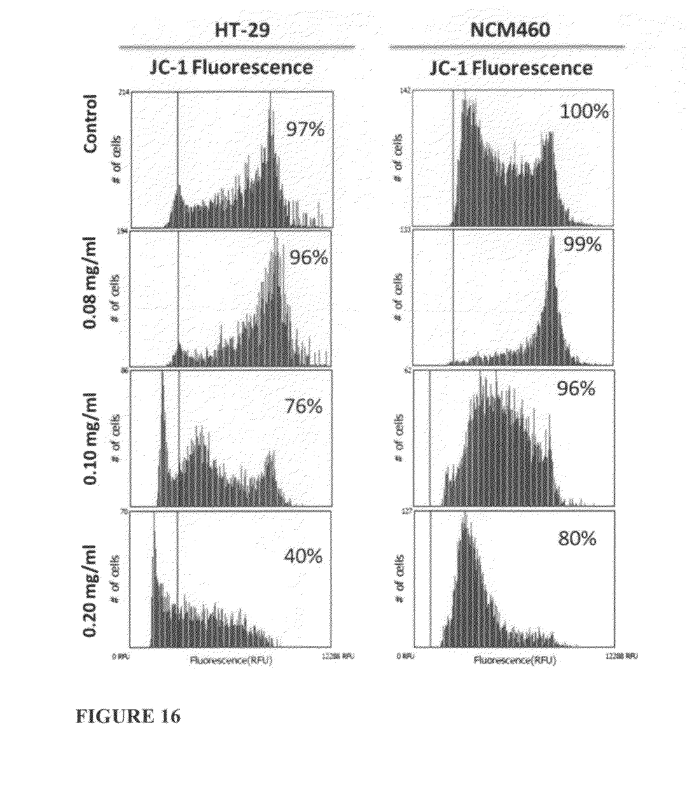

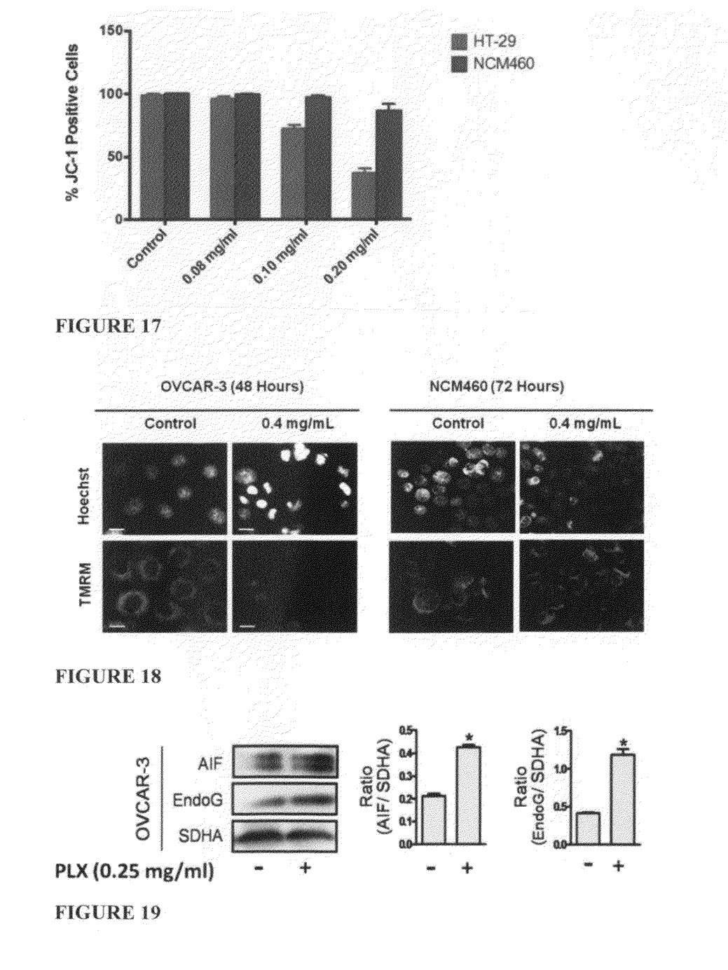

In another experiment, to assess the efficacy of PLX on the mitochondria of cancer cells, OVCAR-3, HT-29 and NCM460 cells were stained with TMRM, a cationic dye that accumulates in healthy mitochondria. Mitochondrial membrane potential (MMP) dissipation was only observed in OVCAR-3 and HT-29 cells as seen with the dissipation of red TMRM fluorescence, by fluorescence microscopy and image-based cytometry (see FIGS. 16 to 18). Following mitochondrial membrane collapse, we wanted to determine if there was release of some pro-apoptotic factors. Western blot analysis was used to monitor for the release of AIF and EndoG from isolated OVCAR-3 mitochondria. Results demonstrate that PLX directly caused the release of both AIF and EndoG from the mitochondria of OVCAR-3 cells (see FIG. 19). These results provide an insight to the mechanism of PLX action, where the mitochondria appears to be a direct target of PLX for the reduction of viability and the induction of apoptosis.

### iv) Long Pepper Extract is Well-Tolerated in Animal Models

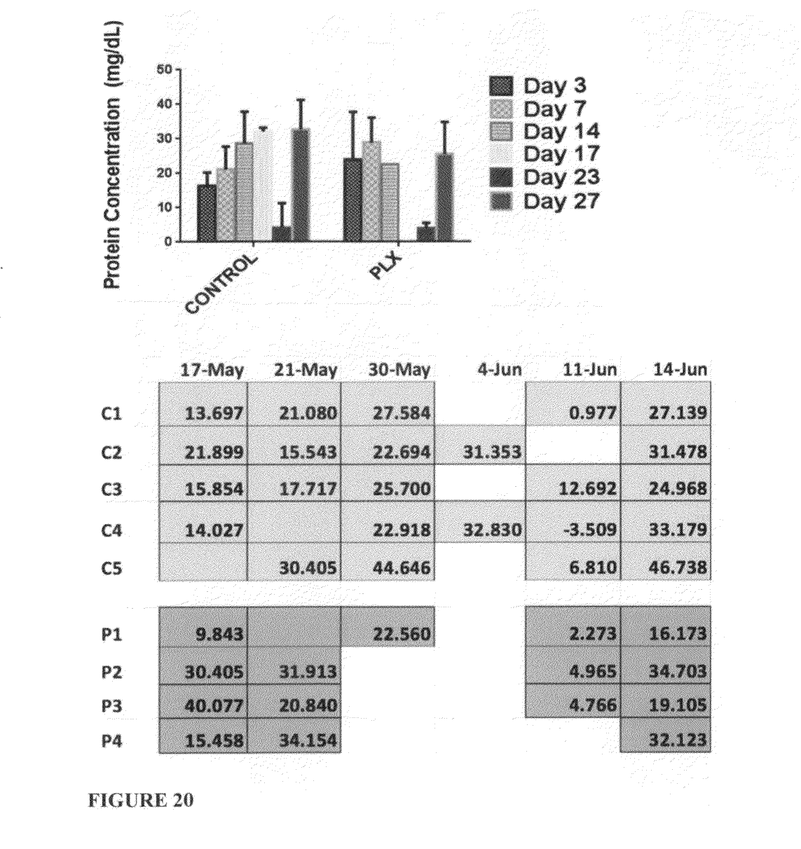

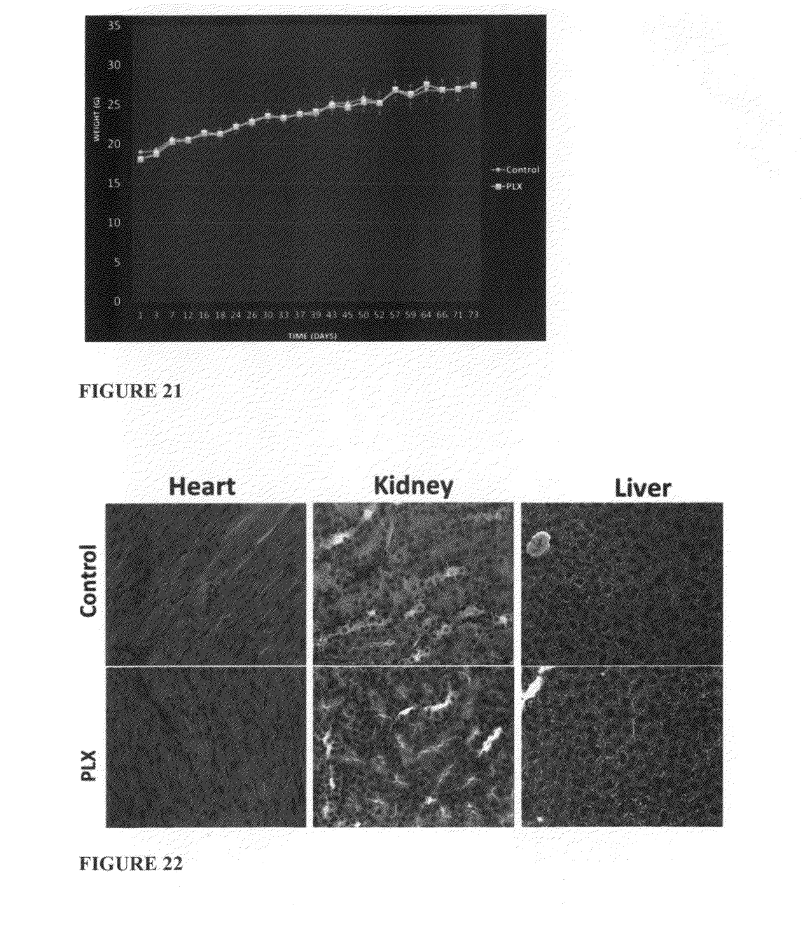

In another experiment, to scientifically evaluate and validate the safety of PLX, balb/c mice were orally gavaged with 50 mg/kg/day vehicle (DMSO) or PLX for 75 days and the mice were observed for signs of toxicity. To assess for toxicity, mice were weighed twice a week, urine was collected for protein urinalysis studies and following period of treatment, mice were sacrificed and their organs were obtained for pathological analysis by a certified pathologist at the University of Guelph (Dr. Brookes). Results from this part of the study demonstrate that there was no weight loss overall in mice that were given PLX supplemented water (see FIG. 21). To further assess toxicity, urine was collected from mice once a week and protein urinalysis was performed using a urine dipstick and a Bradford protein concentration assay. Protein urinalysis results indicate that there were trace amounts of protein in the urine of mice both from the control and the PLX group, with trace readings corresponding to protein concentrations between 5 and 20 mg/dL (data not shown). Bradford assays confirm the results obtained by dipstick urinalysis (see FIG. 20). There was no major difference between the control group and PLX group, confirming the lack of toxicity associated with oral administration of PLX in drinking water. Furthermore, the hearts, livers and kidneys were obtained following the toxicity study, sliced and stained with hematoxylin and eosin. Results show no gross morphologic difference between the control and the treatment group, confirming the lack of toxicity associated with PLX treatment. Results from the pathologist, indicate that the presence of any lesions in the tissues are minimal or mild and interpreted as either background or incidental lesions, and the lack of lesion type and frequency was enough to conclude no toxicological effect of PLX to the balb/c mice (Table 1).

TABLE 1
Summary of Histological Lesions in Balb/C Mice on PLX regimen

| | No | | Vehicle | | | Long | | | |
|---|---|---|---|---|---|---|---|---|---|
| | Treat- | | (Gavage | | | Pepper Extract | | | |
| | ment | | Control) | | | (Treatment group) | | | |
| | M1 | M2 | M1 | M2 | M3 | M1 | M2 | M3 | M4 |
| Liver: | | | | | | | | | |
| Infiltration, leukocyte, | | X | X | | X | X | | | X |
| predominantly | | | | | | | | | |
| mononuclear, minimal | | | | | | | | | |
| Focal mineralization, minimal | | | | | | | | | |
| Hepatocyte necrosis, minimal | | | | | | | | | X |
| Focus of cellular alteration, | | | X | X | | | | X | |
| eosinophilic, minimal | | | | | | | | | |
| Hepatocyte vacuolation, | | | X | X | | | X | | |
| lipid type, minimal | | | | | | | | | |
| Hepatocyte vacuolation, | X | | | X | | | | X | X |
| lipid type, mild | | | | | | | | | |
| Fibrin thrombus | | | X | | | | | | |
| Heart: | | | | | | | | | |
| Infiltration, leukocyte, | | X | | | | X | | | X |
| predominantly | | | | | | | | | |
| mononuclear, minimal | | | | | | | | | |
| Myofiber separation | | X | X | | | | | | X |
| and vaculation, | | | | | | | | | |
| minimal (suspect artifact) | | | | | | | | | |
| Kidney: | | | | | | | | | |
| Infiltration, leukocyte, | X | X | | X | | X | | X | |
| predominantly mononuclear, | | | | | | | | | |
| minimal | | | | | | | | | |
| Tubule vacuolation, minimal | | | | | X | | | | X |
| Fibrin or other extracellular | | | | | | | | X | |
| matrix, glomerulus | | | | | | | | | |

### v) Oral Administration of Long Pepper Extract Halts the Growth of Human Colon Cancer Xenografts in Immunocompromised Mice

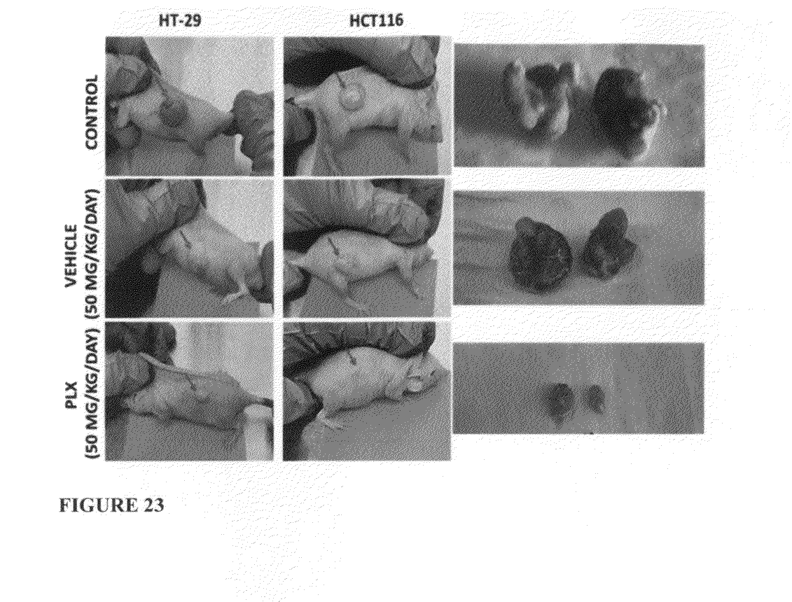

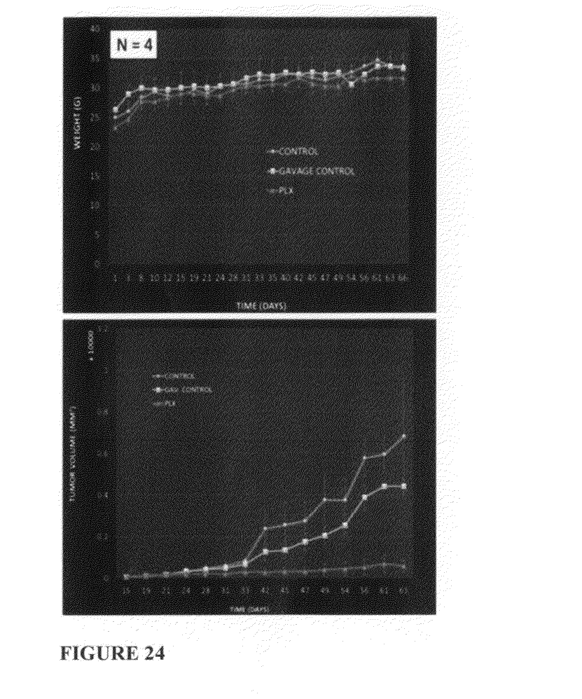

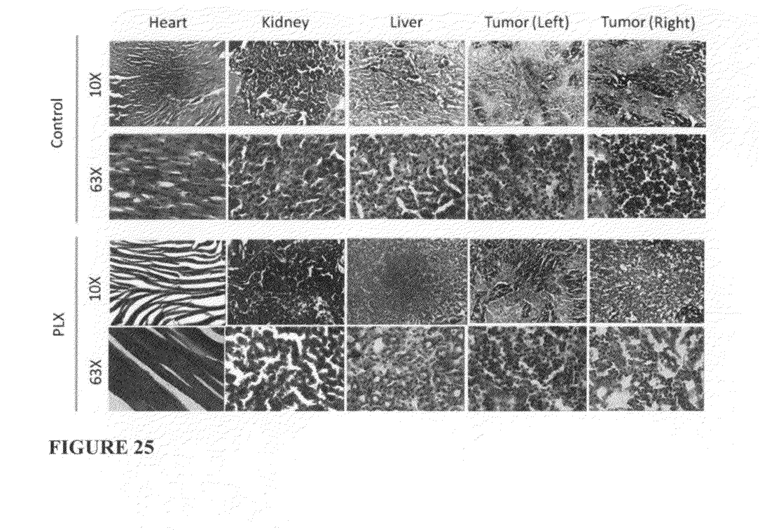

In another experiment to study the efficacy of PLX, CD-1nu/nu immunocompromised mice were subcutaneously injected with HT-29 cells (left) and HCT116 cells (right). Following the establishment of tumors, mice were separated into three groups, a control group, a vehicle (Me₂SO) group and a PLX treated group. Mice were observed for 75 days, with weights and tumor volumes measured twice a week. Results demonstrate that oral administration of PLX could suppress the growth of both p53 WT (HCT116) and p53 mutant (HT-29) tumors in-vivo. There were no signs of toxicity, as indicated by increasing weights during the study (see FIGS. 23 and 24). Furthermore, H & E staining revealed less nuclei in the PLX treated group, compared to the control group, however, as observed in the toxicity studies, there were no gross morphological differences in the livers, kidneys and hearts of the control and PLX groups (see FIG. 25).

### vi) Analysis of Long Pepper Extract

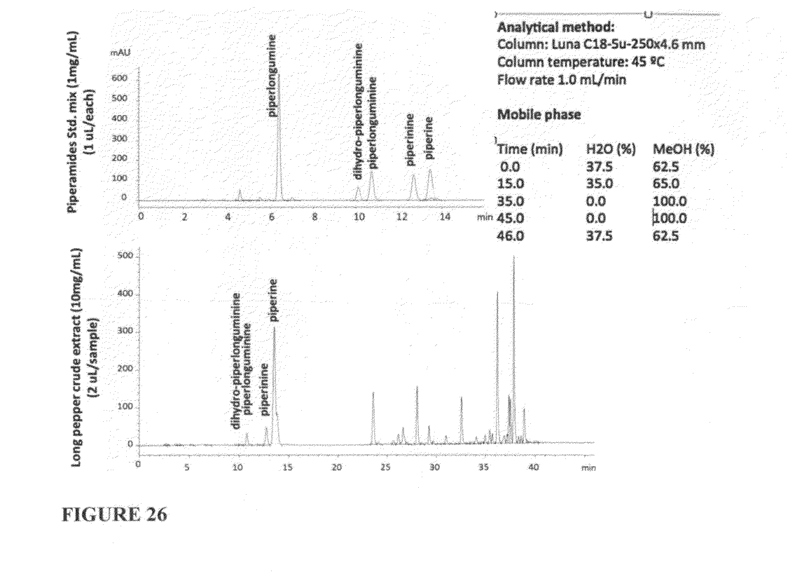

Our collaborators at the University of Ottawa ran an HPLC profile study on the crude ethanolic extracts, compared with a piperamide standard mix. The chromatogram profile showed that our PLX extract contained several classes of compounds known to be present in piper species, including piperines and dihydropiperlongumine. We observed a lack of piperlongumine peaks in our PLX extract (see FIG. 26), suggesting that the PLX may be obtained from the species Piper Belle or the Piper Retrofactum.

The applicant has appreciated the selective anticancer potential of an ethano lie extract of long pepper in several cancer and non-cancerous counterparts. The preferred PLX was shown to effectively reduce the viability of cancer cells, and induced apoptosis in a dose- and time-dependent manner, at low doses, allowing for a greater therapeutic window in in-vitro studies (see FIGS. 1 to 4 and 8 to 10). This apoptosis inducing effect was found to be independent of caspases, cysteine aspartic proteases that play a role in the progression and execution of apoptosis (see FIG. 12). These results suggest that PLX is not toxic to non-cancerous cells at such low doses, as was observed in the cancer cells. Selectivity and lack of toxicity was shown with in-vivo toxicological studies. Furthermore, damage to the kidneys is a common occurrence during various types to toxic therapies. This damage to the kidney results in large amounts of protein (>3.5 g/day) leaking into the urine, and this can be measured by various assays. Lack of toxicity was shown by the lack of increased protein concentration in the urine samples collected from both the control group and PLX treated group, by two different assays. The urine dipstick method indicated that all urine samples from the control and PLX groups had trace amounts of protein, corresponding to concentrations between 5 mg/dL and 20 mg/dL, well within the acceptable concentration range. Bradford protein assay showed a concentration of approximately 30 mg/dL most days urine was collected (see FIG. 20). This is still within the acceptable range of protein concentration in urine. These results suggest reduced associated toxicity or side effects observed with take long pepper extracts. The efficacy of PLX in in-vivo models also showed that not only was PLX well-tolerated, it was also effective at halting the growth of human tumor xenografts of colon cancer in nude mice (see FIGS. 20 and 21).

The next step in understanding the effect of PLX on cell death induction in cancer cells was to identify the mechanism of apoptosis induction observed following PLX treatment. The role of oxidative stress in cell death processes has been well characterized. It is well established the reactive oxygen species (ROS) could be the cause or effect of apoptosis induction in cells. Some studies have suggested cancer cells to be more dependent on cellular response mechanisms against oxidative stress and have exploited this feature to selectively target cancer cells. The role of ROS generation in PLX-induced apoptosis was assessed following treatment. In this study, we found that PLX induced whole cell ROS production in a dose dependent manner, as indicated by the increase in green fluorescence of H₂DCFDA dye, cleaved by intracellular esterases and oxidized by ROS present (see FIGS. 13 and 14). However, we observed that ROS generation was not completely essential to PLX activity, as the presence of N-acetylcysteine could not entirely hamper the ability of PLX to reduce the viability of colon cancer cells (see FIG. 15).

The caspase-independence observed in FIG. 12 suggests that PLX is acting through pro-apoptotic factors other than caspases. The mitochondria play a major role in the progression and execution of apoptosis. The permeabilization of the mitochondrial membrane usually leads to the release of pro-apoptotic factors, including cytochrome c, apoptosis inducing factor (AIF) and endonuclease G (EndoG). AIF and EndoG execute apoptosis in a caspase-independent possibly leading to the caspase- and partial ROS-independence observed. PLX was shown to cause MMP dissipation in cancer cells, while non-cancerous NCM460 cell mitochondria remained intact following treatment (see FIGS. 16 to 18). The dissipation of the mitochondrial membrane led to the release of AIF and EndoG (see FIG. 19), allowing for the progression and execution of apoptosis in the absence of caspases and oxidative stress, providing insight to the mechanism of PLX action in cancer cells. Cancer cells differ from non-cancerous cells in variety of ways, which could enhance the selectivity of PLX to cancer cells. The Warburg effect is characterized by the high dependence of cancer cells on glycolysis and low dependence on mitochondria for energy production in cancer cells, therefore creating a more vulnerable target in cancer cell mitochondria. Moreover, various anti-apoptotic proteins associated to the mitochondria have been reported to be highly expressed in cancer cells. Such proteins could serve as targets for selective cancer.

It has been appreciated that unlike isolated natural compounds, there may be more benefits to using a whole plant extract than the isolated compound. Multiple components within extracts have many different intracellular targets, which may act in a synergistic way to enhance specific activities (including anticancer activities), while inhibiting any toxic effects of one compound alone. Additionally, the presence of multiple components may possibly decrease the chances of developing chemoresistance. Moreover, natural extracts can be administered orally to patients, as a safe mode of administration. Some known compounds of the long pepper plants have been isolated and studied for their various activities. It has been shown that there are some signature compounds that are present in the preferred PLX extract, including dihydropiperlongumine and piperine. Notably, piperlongumine, a compound from the Piper longum plant, has previously been shown to have selective anticancer activity. The preferred ethanolic extract did not contain piperlongumine as seen in the HPLC chromatogram in FIG. 26, as piperlongumine may not have been well extracted or the compound may have been reduced to the dihyropiperlongumine peak that we observe. In a previous study that showed the efficacy of piperlongumine, high concentrations of 10 was required for significant cell death induction in cancer cells. In this study, a very low amount of the complex mixture of the ethanolic extract of long pepper (that contains many bioactive compounds) was shown to be sufficient in inducing apoptosis in cancer cells selectively. This indicates that the individual bioactive compounds (present in nanomolar concentrations within the extract) could act synergistically to induce apoptosis in cancer cells at very low concentrations. These findings highlights that the Piper spp. may contain novel compounds with potent anticancer activity, in addition to piperlongumine.

The studies described herein suggest that a long pepper extract may advantageously be selective in inducing cell death in cancer cells by targeting non-genomic targets. It appears to be well tolerated in mice models and effective in reducing the growth of human tumor xenotransplants in animal models, when delivered orally. The present invention may thus provide for the development of a novel cancer treatment, using complex natural health products from the long pepper.

The following provides for detailed description of the experimental materials and methods used for obtaining the results of the experiments detailed above:

### i) Cell Culture

A malignant melanoma cell line G-361, human colorectal cancer cell lines HT-29 and HCT116 (American Type Culture Collection, Manassas, Va., USA Cat. No. CRL-1687, CCL-218 & CCL-247, respectively) were cultured with McCoy's Medium 5a (Gibco BRL, VWR, Mississauga, ON, Canada) supplemented with 10% (v/v) FBS (Thermo Scientific, Waltham, Mass., USA) and 10 mg/ml gentamicin (Gibco, BRL, VWR). The ovarian adenocarcinoma cell line OVCAR-3 (American Type Culture Collection, Cat. No. HTB-161) was cultured in RPMI-1640 media (Sigma-Aldrich Canada, Mississauga, ON, Canada) supplemented with 0.01 mg/mL bovine insulin, 20% (v/v) fetal bovine serum (FBS) standard (Thermo Scientific, Waltham, Mass., USA) and 10 mg/mL gentamicin. The pancreatic adenocarcinoma cell line BxPC-3 (American Type Culture Collection, Cat. No. CRL-1424) was cultured in RPMI-1640 medium, supplemented with 10% (v/v) fetal bovine serum (FBS) standard and 10 mg/mL gentamicin. Normal-derived colon mucosa NCM460 cell line (INCELL Corporation, LLC., San Antonio, Tex., USA) was grown in INCELL's M3Base™ medium (INCELL Corporation, LLC., Cat. No. M300A500) supplemented with 10% (v/v) FBS and 10 mg/mL gentamicin.

All cells were grown in optimal growth conditions of 37° C. and 5% CO2. Furthermore, all cells were passaged for ≦6 months.

### ii) Cell Treatment

Cells were plated and grown to 60-70% confluence, before being treated with PLX, N-Acetyl-L-cysteine (NAC) (Sigma-Aldrich Canada, Cat. No. A7250), and broad-spectrum caspase inhibitor, Z-VAD-FMK (EMD Chemicals, Gibbstown, N.J., USA) at the indicated doses and durations. NAC was dissolved in sterile water. Z-VAD-FMK was dissolved in dimethylsulfoxide (Me₂SO). PLX was extracted as previously described, reconstituted in DMSO and cells were treated either crude long pepper extract, before evaporation or Me₂SO reconstituted extract and control cells were treated with corresponding concentrations of Me₂SO.

iii) Cell Treatment

Cells were plated and grown to 60-70% confluence, before being treated with PLX, N-Acetyl-L-cysteine (NAC) (Sigma-Aldrich Canada, Cat. No. A7250), and broad-spectrum caspase inhibitor, Z-VAD-FMK (EMD Chemicals, Gibbstown, N.J., USA) at the indicated doses and durations. NAC was dissolved in sterile water. Z-VAD-FMK was dissolved in dimethylsulfoxide (Me₂SO). PLX was extracted as previously described, reconstituted in DMSO and cells were treated either crude long pepper extract, before evaporation or Me₂SO reconstituted extract and control cells were treated with corresponding concentrations of Me₂SO.

### iv) Assessing the Efficacy of Long Pepper Extract (PLX) in Cancer Cells:

WST-1 Assay for Cell Viability

To assess the effect of PLX on cancer cells, a water-soluble tetrazolium salt (WST-1) based colorimetric assay was carried out as per manufacturer's protocol (Roche Applied Science, Indianapolis, Ind., USA) to quantify cell viability as a function of cellular metabolism. Equal number of cells were seeded onto 96-well clear bottom tissue culture plates then treated with the indicated treatments at the indicated concentrations and durations. Following treatment, cells were incubated with the WST-1 reagent for 4 hours at 37° C. with 5% CO₂. The WST-1 reagent is cleaved to formazan by cellular enzymes in actively metabolizing cells. The formazan product was quantified by taking absorbance readings at 450 nm on a Wallac Victor™ 1420 Multilabel Counter (PerkinElmer, Woodbridge, ON, Canada). Cellular viability was expressed as percentages of the solvent control groups.

Nuclear Staining

Subsequent to treatment, the nuclei of cells were stained with Hoechst 33342 dye (Molecular Probes, Eugene, Oreg., USA) to monitor nuclear morphology for apoptosis induction at designated time points. Cells were incubated with 10 μM Hoechst dye for 10 minutes and micrographs were taken with a Leica DM IRB inverted fluorescence microscope (Wetzlar, Germany) at 400× magnification.

Annexin V Binding Assay

To confirm the induction of apoptosis, the binding of Annexin V to externalized phosphatidylserine on the outer cellular surface, was assessed. Following treatment with PLX, cells were washed twice in phosphate buffer saline (PBS). Subsequently, cells were resuspended and incubated in Annexin V binding buffer (10 mM HEPES, 10 mM NaOH, 140 mM NaCl, 1 mM CaCl2, pH 7.6) with Annexin V AlexaFluor-488 (1:50) (Invitrogen, Canada, Cat No. A13201) for 15 minutes. Micrographs were taken at 400× magnification on a Leica DM IRB inverted microscope (Wetzlar, Germany).

Whole Cell ROS Generation

Following treatment with PLX, cells were incubated with 2′,7′-Dichlorofluorescin diacetate H₂DCFDA (Catalog No. D6883, Sigma Aldrich, Mississauga ON. Canada) for 45 minutes. Cells were collected, washed twice in PBS and green fluorescence was observed using a TALI image-based cytometer (Invitrogen, Canada). NAC was used to assess the dependence of PLX on ROS generation and viability.

### v) Assessment of Mitochondrial Function Following PLX Treatment:

Tetramethylrhodamine Methyl Ester (TMRM) Staining

To monitor mitochondrial membrane potential (MMP), tetramethylrhodamine methyl ester (TMRM) (Gibco BRL, VWR, Mississauga, ON, Canada) or 5,5,6,6′-tetrachloro-1,1′,3,3′-tetraethylbenzimidazolylcarbocyanine chloride (JC-1) (Invitrogen, Canada) were used. Cells were grown on coverslips, treated with the indicated concentrations of treatments at the indicated time points, and incubated with 200 nM TMRM for 45 minutes at 37° C. Micrographs were obtained at 400× magnification on a Leica DM IRB inverted fluorescence microscope (Wetzlar, Germany). To confirm the results obtained by fluorescence microscopy, image-based cytometry was used to detect red fluorescence. Cells were seeded in 6-well plates and following treatment, cells were incubated with TMRM for 45 minutes, washed twice in PBS and placed in TALI slides. Red fluorescence was obtained using a TALI image-based cytometer (Invitrogen, Canada).

Mitochondrial Isolation to Assess Mitochondrial Targeting

Cells were collected by trypsin, washed once in cold PBS, resuspended in cold hypotonic buffer (1 mM EDTA, 5 mM Tris-HCl, 210 mM mannitol, 70 mM sucrose, 10 μM Leu-pep and Pep-A, 100 μM PMSF), and manually homogenized. The homogenized cell solution was centrifuged at 3000 rpm for 5 minutes at 4° C. The supernatant was centrifuged at 12,000 rpm for 15 minutes at 4° C. and the mitochondrial pellet was resuspended in cold reaction buffer (2.5 mM malate, 10 mM succinate, 10 μM Leu-pep and Pep-A, 100 μM PMSF in PBS). The isolated mitochondria were treated with PLX at the indicated concentrations and incubated for 2 hours in cold reaction buffer. The control group was treated with solvent (ethanol). Following 2 hour incubation with extract, mitochondrial samples were vortexed and centrifuged at 12,000 rpm for 15 minutes at 4° C. The resulting supernatant and mitochondrial pellets (resuspended in cold reaction buffer) were subjected to Western Blot analysis to assess for the mitochondrial release/retention of pro-apoptotic factors.

Western Blot Analyses

Protein samples were subjected to SDS-PAGE, transferred onto a nitrocellulose membrane, and blocked with 5% w/v milk TBST (Tris-Buffered Saline Tween-20) solution for 1 hour. Membranes were incubated overnight at 4° C. with an anti-endonuclease G (EndoG) antibody (1:1000) raised in rabbits (Abeam, Cat. No. ab9647, Cambridge, Mass., USA), an anti-succinate dehydrogenase subunit A (SDHA) antibody (1:1000) raised in mice (Santa Cruz Biotechnology, Inc., sc-59687, Paso Robles, Calif., USA), or an anti-apoptosis inducing factor (AIF) antibody raised in rabbits (1:1000) (Abeam, Cat. No. ab1998, Cambridge, Mass., USA). After primary antibody incubation, the membrane was washed once for 15 minutes and twice for 5 minutes in TBST. Membranes were incubated for 1 hour at room temperature with an anti-mouse or an anti-rabbit horseradish peroxidase-conjugated secondary antibody (1:2000) (Abeam, ab6728, ab6802, Cambridge, Mass., USA) followed by three 5-minute washes in TBST. Chemiluminescence reagent (Sigma-Aldrich, CPS160, Mississauga, ON, Canada) was used to visualize protein bands and densitometry analysis was performed using ImageJ software.

vi) In-Vivo Assessment of Long Pepper Extract

Toxicity Assessment

Six week old Balb/C mice were obtained from Charles River Laboratories and housed in constant laboratory conditions of a 12-hour light/dark cycle, in accordance with the animal protocols outlined in the University of Windsor Research Ethics Board—AUPP 10-17). Following acclimatization, mice were divided into three groups (3 animals/control (untreated), 3 animals/gavage control (vehicle treatment) and 4 animals/treatment group). The control untreated group was given plain filtered water, while the second and third group was given 50 mg/kg/day vehicle (DMSO) or PLX, respectively for 75 days. During the period of study, toxicity was measured by weighing mice twice a week and urine was collected for protein urinalysis by urine dipstick and Bradford assays. Following the duration of study, mice were sacrificed and their organs (livers, kidneys and hearts) were obtained for immunohistochemical and toxicological analysis by Dr. Brooke at the University of Guelph.

Efficacy of PLX in Tumor Xenograft Models of Immunocompromised Mice

Six week old male CD-1 nu/nu mice were obtained from Charles River Laboratories and housed in constant laboratory conditions of a 12-hour light/dark cycle, in accordance with the animal protocols outlined in the University of Windsor Research Ethics Board—AUPP 10-17). Following acclimatization, the mice were injected subcutaneously in the right and left hind flanks with a colon cancer cell suspension (in Phosphate buffered saline) at a concentration of 2*10⁶ cells/mouse (HT-29, p53^{-/-}, in the left flank and HCT116, p53^{+/+}, in the right flank).

Tumors were allowed to develop (approximately a week), following which the animals were randomized into treatment groups of 4 mice per group, a control group, a gavage control group given plain filtered sterile water, as well as gavage regimen of the vehicle (5 μL Me₂SO in PBS) twice a week. The final group was given filtered water supplemented with long pepper extract at a concentration of 100 μg/mL, as well as gavage regimen of long pepper extract (5 extract in PBS), twice a week, corresponding to 50 mg/kg/day. The tumors were assessed every other day by measuring the length, width and height, using a standard caliper and the tumor volume was calculated according to the formula π/6*length*width. The mice were also assessed for any weight loss every other day for the duration of the study, which lasted 75 days, following which the animals were sacrificed and their organs and tissues (liver, kidneys, heart and tumors) were obtained and stored in 10% formaldehyde for immunohistochemical and toxicological analysis.

Hematoxylin & Eosin (H & E) Staining

Mice organs were fixed in 10% formaldehyde, following which they were cryosectioned into 10 □m (μm/mm) sections and placed on a superfrost/Plus microscope slides (Fisherbrand, Fisher Scientific). Sections of organs were stained according to a standardized H & E protocol.

vii) Analysis of Long Pepper Extract by HPLC

HPLC analysis of the long pepper crude extract was carried out at University of Ottawa in the Arnason lab. A total of five well-known piperamides were analyzed and compared to the crude long pepper extract. The extracts and piperamide standards were analyzed on a Luna C18-5u-250×4.6 mm column at 45° C. at a flow rate of 1.0 mL/min with a mobile phase constituted of H₂O and methanol as outlined below;

| Time (mins) | H20 (%) | MeOH (%) |
|---|---|---|
| 0.0 | 37.5 | 62.5 |
| 15.0 | 35.0 | 65.0 |
| n35.0 | 0.0 | 100.0 |
| 45.0 | 0.0 | 100.0 |
| 46.0 | 37.5 | 62.5 |

Chromatogram profiles were used to detect the any differences between a sample standard of known piperamides in the crude long pepper extracts.

Other anticancer ingredients or drugs, which do not impair the functions of the PLX may be added to the medicament of the present invention. Such anticancer ingredients may include, but not limited to, an antifolate, a 5-fluoropyrimidine (including 5-fluorouracil), a cytidine analogue such as β-L-1,3-dioxolanyl cytidine or β-L-1,3-dioxolanyl 5-fluorocytidine, antimetabolites (including purine antimetabolites, cytarabine, fudarabine, floxuridine, 6-mercaptopurine, methotrexate, and 6-thioguanine), hydroxyurea, mitotic inhibitors (including CPT-11, Etoposide (VP-21), taxol, and vinca alkaloids such as vincristine and vinblastine), an alkylating agent (including but not limited to busulfan, chlorambucil, cyclophosphamide, ifofamide, mechlorethamine, melphalan, and thiotepa), nonclassical akylating agents, platinum containing compounds, bleomycin, an anti-tumor antibiotic, an anthracycline such as doxorubicin and dannomycin, an anthracenedione, topoisomerase II inhibitors, hormonal agents (including but not limited to corticosteriods (dexamethasone, prednisone, and methylprednisone), androgens such as fluoxymesterone and methyltestosterone), estrogens such as diethylstilbesterol, antiestrogens such as tamoxifen, LHRH analogues such as leuprolide, antiandrogens such as flutamdie, aminogluetethimide, megestrol acetate, and medroxyprogesterone, asparaginase, carmustine, lomustine, hexamethyl-melamine, dacarbazine, mitotane, streptozocin, cisplatin, carboplatin, levamasole, and leucovorin. Preferably, the anticancer agent is metformin, hydroxyurea, cyclophosphamide or etoposide. The extract of the present invention can also be used in combination with enzyme therapy agents and immune system modulators such as an interferon, interleukin, tumor necrosis factor, macrophage colony-stimulating factor and colony stimulating factor.

The PLX may be administered to a patient by any appropriate route which, for example, may include oral, parenteral, intravenous, intradermal, transdermal, mucosal, subcutaneous, and topical. Preferably, the root extract is administered orally. A number of administration/dosage experiments showed that the medicament of the present invention may produce greater anticancer activity if ingested orally, and possibly exposed to the subject's digestive system. The extract may be orally administered in powder or liquid extract form without further modifications. Alternatively, the extract may be solubilized in a liquid, most preferably in water, the liquid containing the extract is orally administered. The extract may alternatively be enclosed in capsules or compressed into tablets. Such capsules or tablets may be purified to remove impurities and/or bacteria, or further include an inert diluent, an edible carrier, binding agents, and/or adjuvant materials.

The tablets, capsules, and the like can contain any of the following ingredients, or compounds of similar nature: a binder such as microcrystalline cellulose, gum tragacanth or gelatin; an excipient such as starch or lactose; a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to the aforementioned materials, a liquid carrier such as fatty oil. In addition, dosage unit forms can contain various other materials which modify the physical form of the dosage unit, for example, coating of sugar, shellac, or other enteric agents.

It is to be noted that dosage will vary with the conditions, age, body weight and severity of the cancer to be treated. It will be readily apparent to a person skilled in the art that for each patient, specific dosage regimens could be adjusted over time according to individual needs. The extract may be administered once or may be divided into a number of smaller doses to be administered at varying intervals of time.

The medicament of the present invention is suitable for treatment and/or prevention of a cancer, including that of skin tissues, organs, bone, cartilage, blood and vessels. The root extract may be used to treat variety of cancers including, but not limited to, cancer of the head, neck, eye, mouth, throat, esophagus, chest, bone, lung, colon, rectum, stomach, prostate, breast, ovaries, kidney, liver, pancreas and brain. The cancer encompasses primary and metastatic cancers.

The most preferred embodiments of the present invention are described hereto. The most preferred embodiments are provided as mere examples which are in no way intended to limit the scope of the present invention. It will be readily apparent to a person skilled in the art that variations and modifications may be made to the most preferred embodiments within the scope of the present invention.

1. A method for preparing a medicament for the treatment or prevention of a cancer, the method comprising:
grinding a Piper plant or a plant component thereof to obtain a ground plant mixture or powder;
soaking the ground plant mixture or powder in a solvent to obtain a suspension having a liquid extract portion and a plant solid portion; and
separating the liquid extract portion from the plant solid portion to provide a separated liquid extract for use in the medicament.

2. The method of claim 1, wherein the plant component comprises one or more of a seed, a leaf, a flower, a fruit, a root and a stem.

3. The method of claim 1, wherein the solvent comprises one or more of water, pentane, cyclopentane, cyclohexane, benzene, toluene, 1,4-dioxane, chloroform, diethyl ether, dichloromethane, tetrahydrofuran, ethyl acetate, acetone, dimethylformamide, acetonitrile, dimethyl sulfoxide, propylene carbonate, formic acid, alcohol and acetic acid.

4. The method of claim 1, wherein the solvent is ethanol.

5. The method of claim 1, wherein said soaking step comprises soaking the ground plant mixture or powder with or without shaking in the solvent for between about 5 minutes and about 72 hours, and at a temperature between about 0° C. and about 100° C.

6. The method of claim 1, wherein the method further comprises freezing the Piper plant or the plant component to obtain a frozen plant stock, said freezing step being selected to effect at least partial disruption of one or more plant cells, and wherein said grinding step comprises dry grinding the frozen plant stock to obtain the ground plant powder, wherein during said dry grinding step, the frozen plant stock is maintained at a grinding temperature below about 40° C.

7. The method of claim 6, wherein said grinding temperature is below about 0° C.

8. The method of claim 6, wherein prior to said freezing step, the method further comprises drying said Piper plant or the plant component to a relative humidity between about 5% and about 10%.

9. The method of claim 6, wherein said freezing step comprises contacting or submerging the Piper plant or the plant component in liquid nitrogen, or freezing the Piper plant or the plant component to an average freezing temperature between about −210° C. and about −30° C.

10. The method of claim 6, wherein said dry grinding step comprises dry grinding the frozen plant stock to an average particle size of less than about 100 μm.

11. The method of claim 6, wherein said dry grinding step comprises dry grinding the frozen plant stock with a grinder selected from the group consisting of a pulverizer, an impingement grinder and a micronized milling machine, and wherein the grinder or a component thereof is cooled below about −25° C. to prevent heating on contact with the frozen plant stock or the ground plant powder.

12. The method of claim 11, wherein the grinder or the component is cooled by directly or indirectly contacting with liquid nitrogen.

13. The method of claim 12, wherein the grinder defines a grinding chamber sized for receiving the frozen plant stock, said method further comprising flowing or adding liquid nitrogen to the grinding chamber during said dry grinding the frozen plant stock in the grinding chamber.

14. The method of claim 11, wherein said dry grinding step further comprises straining the ground plant powder from the grinder through a sieve sized to obtain a sieved ground plant powder having an average particle size of less than about 100 μm.

15. The method of claim 1, wherein said separation step comprises filtration, and wherein said filtration is performed once or more than once using a plurality of filters of same or different pore sizes.

16. The method of claim 1, wherein said separation step comprises filtering the suspension at least twice with a paper filter having a particle retention greater than about 20 μm.

17. The method of claim 1, wherein said method further comprises removing the solvent from the separated liquid extract to obtain a solid or semi-solid extract, and optionally mixing the extract with one or more of a pharmaceutically acceptable carrier, a diluent, a binding agent, an adjuvant and an anticancer agent.

18. The method of claim 17, wherein the carrier comprises one or more of a polar reconstitution solvent and a buffer solution.

19. The method of claim 17, wherein the anticancer agent comprises metformin, hydroxyurea, cyclophosphamide or etoposide.

20. The method of claim 1, wherein the Piper plant comprises one or more of Piper longum, Piper belle, Piper nigrum and Piper retrofactum.

21. The method of claim 1, wherein the cancer is colon cancer, colorectal cancer, ovarian cancer, pancreatic cancer, melanoma, breast cancer, osteosarcoma, lung cancer, prostate cancer, glioblastoma, lymphoma or leukemia, wherein the leukemia includes T cell leukemia, acute myeloid leukemia, chronic myeloid leukemia, chronic myelomonocytic leukaemia, chronic lymphocytic leukemia or acute lymphoblastic leukemia.

22. The method of claim 1, wherein the separated liquid extract comprises two or more of dihydropiperlongumine, piperlongumine, dihydropiperlonguminine, piperlonguminine, piperinine, piperazine, piperidine and piperine.
